(19) United States
(12) Patent Application Publication
(10) Pub. No.: US 2018/0197638 A1
(43) Pub. Date: Jul. 12, 2018

(54) SECURE SYSTEM FOR A REMOTE HEALTH CARE PROVIDER TO CONSULT WITH A CARE TEAM

(71) Applicant: Sensory Technologies Inc., London (CA)

(72) Inventors: Patrick Blanshard, London (CA); Tom Sobut, London (CA); Andrew Krasnov, London (CA)

(73) Assignee: Sensory Technologies Inc., London (CA)

(21) Appl. No.: 15/867,865

(22) Filed: Jan. 11, 2018

Related U.S. Application Data
(60) Provisional application No. 62/445,150, filed on Jan. 11, 2017.

(30) Foreign Application Priority Data
Jan. 11, 2017 (CA) 2954295

(51) Int. Cl. G16H 80/00; G16H 10/60

(52) U.S. Cl. CPC G16H 80/00; G16H 10/60

(57) ABSTRACT

A secure system for a remote health care provided to consult with a care team is provided. Also provided are related methods. The system and method enables a visiting nurse, technician, or non-licensed medical professional to work independently, in a patient's home, care facility or non-hospital location, while having immediate and real-time access to authorized clinicians, such as registered nurses, physicians or other licensed medical professionals who are updated and familiar with the patient. Additionally, the clinicians or consultants may have real-time patient specific data through the patient's electronic medical record thereby allowing the clinicians or consultants to direct the visiting nurse regarding the patient's care. All data and metadata sent through the system concerning a patient is recorded in the patient's electronic record creating a complete, real-time, and continuous patient record that is auditable and accountable.

### COPYRIGHT AND TRADEMARK NOTICE

A portion of the disclosure of this patent document contains material which is subject to copyright protection. The copyright owner has no objection to the facsimile reproduction of the patent document or the patent disclosure, as it appears in the Patent and Trademark Office patent file or records, but otherwise reserves all copyright rights whatsoever. Trademarks are the property of their respective owners.

### BACKGROUND

The medical field suffers from a shortage of specialist medical personnel. For example, there may be nurses but there is a shortage of palliative specialist nurses to care for palliative patients. Another issue is that registered nurses (RNs) do not have the sufficient numbers to attend to those who are on an outpatient basis nor those who require regular homecare visits. There are an insufficient number of nurses to care for patients who are in their home but would normally be in the hospital. These patients, who may require palliative care, complex care, pediatric care, or a similar level of attention, may normally be in a hospital. Such patients, located in their homes, normally require at least one shift of nurse care, that is, a nurse would be at their location for up to 12 hours a day.

Responsive to this need a distributed health care system was generated to provide a means for a clinician to remotely monitor the health of a patient located in an environment outside of a hospital, such as a home, long term care facility, hospice, etc. An embodiment of this system is presented in US 2012/0290313, which describes a system for distributed health care that uses personal support workers (PSW) and registered, trained medical personnel. Each PSW is equipped with a mobile computing device that is capable of communicating with a main computer. Each registered medical personnel is equipped with a computing device (a monitoring computer) that is capable of communicating with a main computer. At times during a PSW's shift at a patient location, the PSW inputs data to a number of forms on the mobile computing device, each form being related to the patient's physical appearance, medical condition, medication taken or given, and physical parameters, or other actions taken. The data inputted are then transmitted to the main computer, which processes, stores, and archives the data. After processing, the data is reviewed by the registered medical personnel. If the data indicates that actions need to be taken, the medical personnel can issue instructions to the PSW.

This system can be used by others for example, family members, clinicians, technicians, etc. monitoring and/or providing care to a patient located outside of a hospital, to efficiently collect patient data that is relevant to the patient's care plan and health status to efficiently and reliably document the information into the patient's electronic medical record. The individual working out in the field with the patient can be considered to be a mobile user and, in some instances, can be the patient themselves.

Issues can arise, however, when a mobile user, for example a nurse working with a patient in their home needs to consult with someone on the Care Team. Questions can arise that may be related to the patient's health status requiring clinical information and/or external advice, direction or assistance. For example, if a patient's health deteriorates and additional services are required such as physical therapy or hardware such as a specialized bed, it would be useful if the caregiver could efficiently contact the relevant source of information and institute appropriate changes to the care plan.

In another scenario where there is directing nurse supervising a mobile user who is a technician in the patient's home, the directing nurse may need to consult with a physician in order to address something that is happening with the patient, such as a change in medication or concern about a change in symptoms, for example.

Another requirement is that all of this information is effectively documented into the patient electronic medical record (EMR) so that when health care managers are examining resource utilization, they are aware of how the resources are being allocated. For example, if the mobile user is a nurse visiting a patient and conducts a 15 minute consultation with a physician, that information will be efficiently collected and recorded in the patient's EMR.

When working remotely, it can be challenging for the mobile user to be able to easily ascertain who to contact in terms of who is currently assigned to a patient's Care Team and to be able to actually contact the resources required. Many of the Care Team may be working in a busy clinical environment and may find it challenging to prioritize a request from someone working out in the field. Thus, it is easy to envision situations that can arise whereby the caregiver needs a timely consultation and it can be difficult to get the correct attention.

Even if a caregiver can call someone and have a chat—there may be no way to get this information entered into the patient's EMR—especially when everyone is busy. There usually is no place in the patient record to enter this kind of information. Moreover, different agencies and organizations generally have their own patient records.

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also be inventions.

### SUMMARY

What is provided is a method and system for a remote health care provider to securely consult with a care team.

### BRIEF DESCRIPTION OF THE FIGURES

FIG. 1 depicts an example relationship of the parties of the system.

FIG. 2 depicts an embodiment of the system diagramed in FIG. 1.

FIG. 3 depicts an alternate example relationship of the parties of the system.

FIG. 4 depicts an embodiment of the system diagramed in FIG. 3.

FIG. 5A-5D depict example forms as presented on a mobile device.

FIG. 6A-6D depict example forms as presented on a mobile device.

FIG. 7A-7D depict example pages as presented on a workstation.

FIG. 8 depicts the permission/viewing relationship between the directing clinician, the observing clinician and the plurality of technicians.

FIG. 9 depicts an embodiment of the system diagramed in FIG. 8.

FIG. 10 depicts an example work flow diagram of a chat room functionality.

FIG. 11 depicts examples of web and mobile chat room instructions.

FIG. 12 depicts exemplary mobile chat room GUI and instructions.

FIG. 13A illustrates the roles and permissions aspect of for the members of a patient's Care Team. FIG. 13B depicts yet another example of the system.

FIG. 14 illustrates data workflow between members of a Care Team and members of a third party.

FIG. 15 depicts an embodiment of the system diagramed in FIG. 14.

FIG. 16 depicts an embodiment of the system diagramed in FIG. 14.

FIG. 17 depicts an example workflow of a communication between the third party and the system.

FIG. 18 depicts an example workflow for a consultation.

### DETAILED DESCRIPTION

The following detailed description is merely exemplary and is not intended to limit the described embodiments or the application and uses of the described embodiments. As used, the word “exemplary” or “illustrative” means “serving as an example, instance, or illustration.” Any implementation described as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure. The scope of the invention is defined by the claims. For the description, the terms “upper,” “lower,” “left,” “rear,” “right,” “front,” “vertical,” “horizontal,” and derivatives thereof shall relate to the examples as oriented in the drawings. There is no intention to be bound by any expressed or implied theory in the preceding Technical Field, Background, Summary or the following detailed description. It is also to be understood that the devices and processes illustrated in the attached drawings, and described in the following specification, are exemplary embodiments (examples), aspects and/or concepts defined in the appended claims. Hence, dimensions and other physical characteristics relating to the embodiments disclosed are not to be considered as limiting, unless the claims expressly state otherwise. It is understood that the phrase “at least one” is equivalent to “a”. The aspects (examples, alterations, modifications, options, variations, embodiments and any equivalent thereof) are described regarding the drawings. It should be understood that the invention is limited to the subject matter provided by the claims, and that the invention is not limited to the particular aspects depicted and described.

The Basic Design of the System

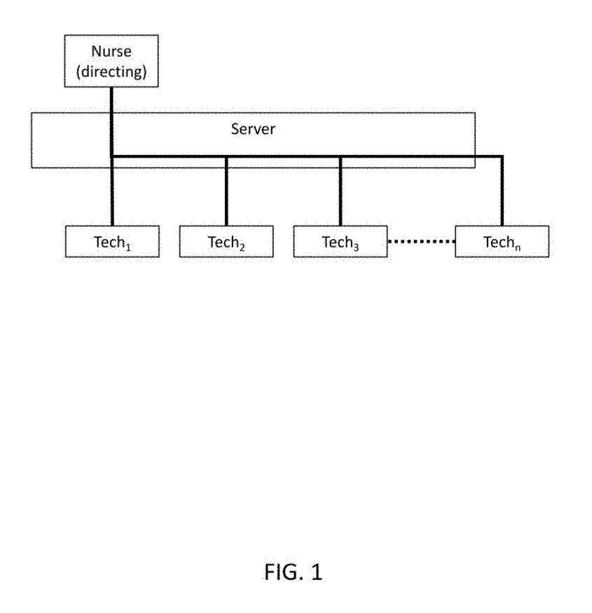

Referring to FIG. 1, a diagram depicting the relationship between a directing clinician (on a directing clinician workstation 2, in this case a nurse) and a mobile user on a mobile device 8 (for example, a technician) providing health care to a patient. A clinician, on a directing terminal 2, is tasked with managing a unique set of selected mobile users on the one or more mobile devices 8 to provide health care to a patient located in a non-hospital facility. All communications between the directing terminal 2 and the mobile devices 8 is managed and facilitated by the server 6. It will be understood that any method of networking can be used to communicatively connect the directing terminal 2, the mobile devices 8, and the server 6 to each other. Examples of networking include, but are not limited to, a VPN, cellular, a LAN, WiFi, etc.

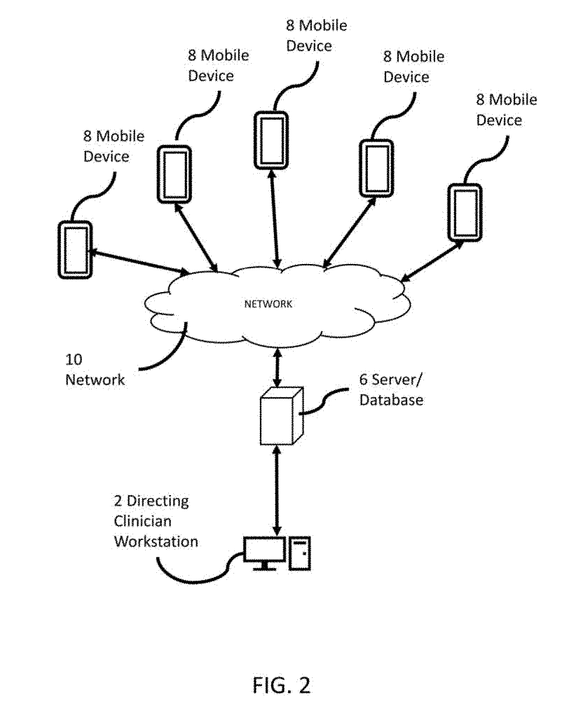

Referring now to FIG. 2, an embodiment of a health care system is illustrated. In this embodiment, the system includes a directing clinician workstation 2 and a plurality of mobile computing devices 8. The directing clinician workstation 2 and the mobile devices 8 are communicatively connected by way of a network through a server 6. Communications between the mobile computing devices and the directing clinician workstation 6 pass through the server 6. This allows the server 6 to process, archive, and store these communications. Communications between the server 6 and any of the directing clinician workstations 2 also pass through a suitable data network. Networks and may be of the same type of data communications network or they may be dissimilar types of communications networks.

The server 6 is configured to intercept, facilitate, process, archive, and/or relay communications between the directing clinician workstation 2 and the plurality of mobile devices 8. The server 6 may include one or more datastores for storing data and metadata. In some examples, the server 6 includes separate databases for storing clinical, patient, and caregiver data. In some examples, the data from one database may be replicated or copied to another database so that the other database contains a subset of data from the first database, such as anonymized data. In another example, the data in each of the databases may also be encrypted.

Each directing clinician workstation 2 is operated by a clinician, including but not limited to a licensed medical professional such as a registered nurse. Each clinician operating a directing clinician workstation 2 observes and manages the users of the mobile devices 8 and the patients cared for by the users of the mobile devices 8.

The mobile computing devices 8 are operated and used by mobile users, who include (but are not limited to) caregivers observing a patient in a location outside of a hospital setting. For example, the patient location may be the patient's home, an outpatient facility, a nursing home, and other non-hospital or non-clinical facility.

Users of the mobile devices 8 can include, but are not limited to, travelling clinicians (e.g., physicians or nurses) visiting patients to conduct check-ups, nurses or technicians (nursing assistants) conducting check-ups or observing the patient over an extended period of time during a shift, non-regulated personnel (such as palliative care volunteers, nursing home general labour, etc, or anyone in a healthcare environment trained to operate the mobile computing devices, care for patients, take health readings such as, for example, blood pressure, pulse, and temperature readings, provide first aid, administer at least some medication, in essence, operating as the eyes, ears, and hands of the observing clinician to the extent that the applicable laws permit). In some instances the mobile user may be a family member, friend of the patient, or the patient themselves.

Referring again to FIG. 2, the clinician who operates the directing clinician workstation 2 observes and manages the mobile users who are using mobile devices 8 while caring for a patient. The server 6 receives all the communications from the various mobile devices 8, processes these communications as well as the data contained in them, including archiving the data, and routes them to the proper directing clinician workstation 2. In the example depicted in FIG. 2, the server 6 and the directing clinician workstations 2 are co-located in the same premises. For example, the server 6 and the directing clinician workstations 2 in this example may be co-located in the same clinical complex, data center, or healthcare facility. In an embodiment, the clinician operates the directing clinician workstation to observe and manage the mobiles user in real-time.

The networks may include the Internet, a dedicated local area network (LAN), a virtual private network (VPN), or any other data network that may be used to communicate and transfer data between two data processing devices.

In some examples, the directing clinician workstation 2 can be a dedicated personal computer, including a laptop, with suitable hardware for communicating with the network or the server 6. The directing clinician workstation 2 may be any computing device capable of communicating with the server 6 via a network such as a tablet, a computing device having a web browser, tablet, or a smartphone.

The system is scalable, wherein in one embodiment, the directing clinician workstation 2 can be one of a plurality of directing clinician workstations 2 with a server 6 to provide access to the various mobile devices 8 in the system. The mobile devices 8 can be any suitable mobile computing device that allows users to access the network, display and receive input into preset forms, and which will allow communications with the directing clinician workstation 2, through the server 6. Smart phones, tablet computers, laptops, and any other such device can be used as one of the mobile computing devices. In some embodiments, the mobile computing devices have touch screen capabilities.

In one embodiment, mobile user may be a nursing assistant or non-regulated person who attends to a patient for a specific shift (e.g., a 6, 8 or 12 hour shift) during which the mobile user provides care to the patient under the management and supervision of the remote clinician on the directing clinician workstation 2. During that shift, the mobile user fills out the requested or required forms on the mobile device 8 for the specific patient. The forms have entries for the various physical parameters of each patient that are appropriate for the patient's condition and their surroundings. The parameters include, but are not limited to, blood pressure, appearance, any symptoms they may have, whether they have taken their medication, etc.), the state of any medical equipment they are using (for example, if they are on a heart monitor, is the heart monitor in good, working condition), the patient's mood, etc.

It will be understood that the system comprising server 6, directing clinician workstation 2, and/or mobile device 8 can be configured with the elements in many different locations, as long as the, directing clinician workstation 2, and mobile devices 8 are securely and effectively communicatively connected with the central server. For instance, in some examples the server 6, one or more directing clinician workstations 2, and one or more mobile devices 8 may be co-located (i.e., in the same physical location, such as a clinical facility or hospice). In other examples the server, one or more directing clinician workstations 2 may be co-located while the mobile devices 8 may be used remotely in another facility such as a patient's home. In yet another example, the server 6, directing clinician workstations 2, and mobile devices 8 may be located remotely from each other in separate facilities.

In another example, the server 6 may be implemented in a cloud computing environment. For instance, the server 6 may be implemented on one or more virtual private servers in a cloud computing environment such as AMAZON EC2, GOOGLE COMPUTE ENGINE or other such system.

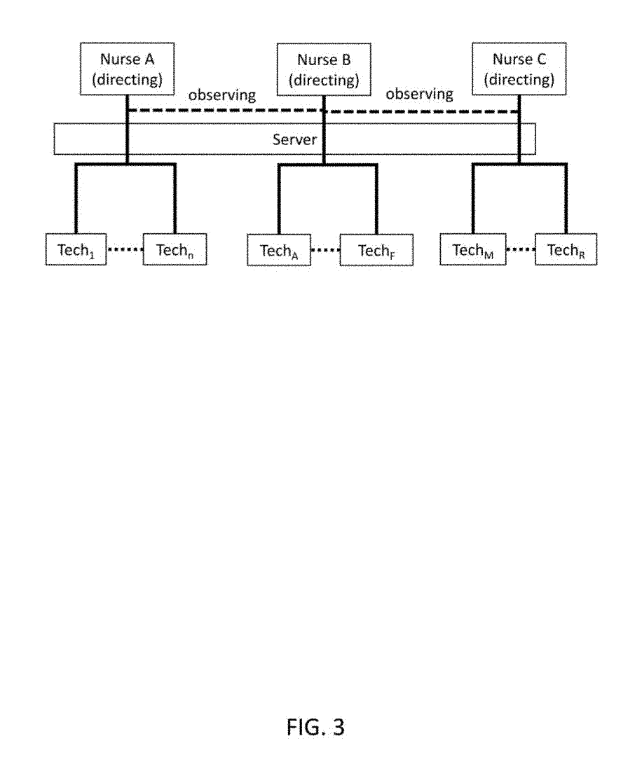

Referring now to FIG. 3, a diagram illustrating an embodiment with an exemplary relationship between one or more clinicians (on directing clinician workstations 2, in this case nurses) and mobile users on a mobile devices 8 (technicians or Techs) is provided. In this example a clinician (a nurse, physician, licensed health care professional, etc.) is responsible for a group of mobile users (in this case technicians) using mobile devices 8. As is depicted in FIG. 3, nurse A is responsible for directing a group of technicians labelled Tech1 to Tech n. Likewise, nurse B is responsible for directing a group of technicians labelled Tech A to Tech F. Finally, clinician N is responsible for directing a group of technicians labelled Tech M to Tech R. Each directing nurse may also select to attain observing status over the other technicians assigned to the other directing nurses. It will be appreciated that the number of mobile devices 8 assigned to a particular directing terminal 2 (in this case, a nurse or clinician in front of a directing terminal 2) may depend on, among other things, the ability of the clinician, the clinician's license, the clinician's workload, the condition of the patients being monitored by the technicians, etc. In an embodiment, the clinician attains observing status of the technician in real-time, and the observing of the technician by the clinician happens in real-time. Real-time is defined as near real-time or soft real-time. Real-time is about 3-6 seconds of delay due to processing by embodiments of the system and method. It is understood that as processing power increases the time delay may decrease. It is also understood that delays due to communication channel performance (e.g. wireless internet, cellular networks, network congestion) may affect the delay and responsiveness of the embodied systems and methods.

In some example embodiments, a mobile user such as a technician on a mobile device 8, for example, may be observed by more than one clinician, with only one on a directing terminal 2 and others on observing clinician workstations so that the technician on the mobile device 8 may be directly and indirectly overseen by more than one clinician. A technician on a mobile device can only be directed by one clinician. Any number of clinicians may observe the current activity of directing clinicians. One of these “observers” can take over direction on the patient if the situation requires. In an embodiment, the take over or transfer of the technician by an observing clinician happens in real-time. The clinician changes roles from an observer to a directing clinician in real-time, and the current directing clinician is removed from the role of directing clinician in real-time.

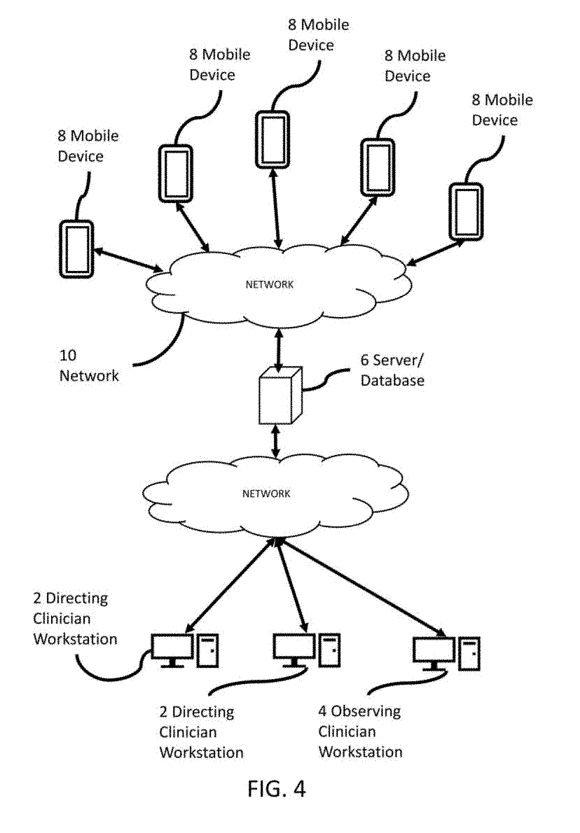

Referring now to FIG. 4, an embodiment of the system diagramed in FIG. 3 is depicted. In this example, the server 6 is deployed in a location that is remote from both the mobile devices 8 and the directing clinician workstation 2. In this example, the server 6 may be connected to the mobile devices 8 over a cellular, WiFi, WiLAN, LAN, VPN, or any other network. Similarly, the directing clinician workstation 2 may be connected to the server 6 over any kind of network (as described above). Similar to FIG. 2, the directing clinician workstation 2 communicates with the mobile device 8 through the server 6.

FIG. 4 illustrates an embodiment comprising the configuration depicted by FIG. 3, wherein the clinician at directing clinician workstation 2 oversees the mobile users of the mobile devices 8, while the clinicians at the observing clinician workstations have observing status vis-à-vis the mobile users assigned to the clinician at the directing clinician workstation 2.

The Mobile Device and Mobile User Forms

In embodiments where a mobile user, such as a technician, works a shift, at the beginning of each shift, the mobile user takes readings of the patient's physical parameters (for example, the patient's vital signs). These readings are then transmitted to the monitoring computer where the readings are provided to the clinician. The other readings are also to be presented to the clinician. In an embodiment, the readings are provided to the clinician in real-time.

Multiple categories of readings for the patient are also taken. Readings related to the patient's vital signs, neurology, respiratory system, cardiovascular system, skin integrity, and gastrointestinal system are taken by the mobile user and the readings are sent to the monitoring computer. These readings are then reviewed by the clinician at the directing clinician workstation to ensure that they are within acceptable parameters

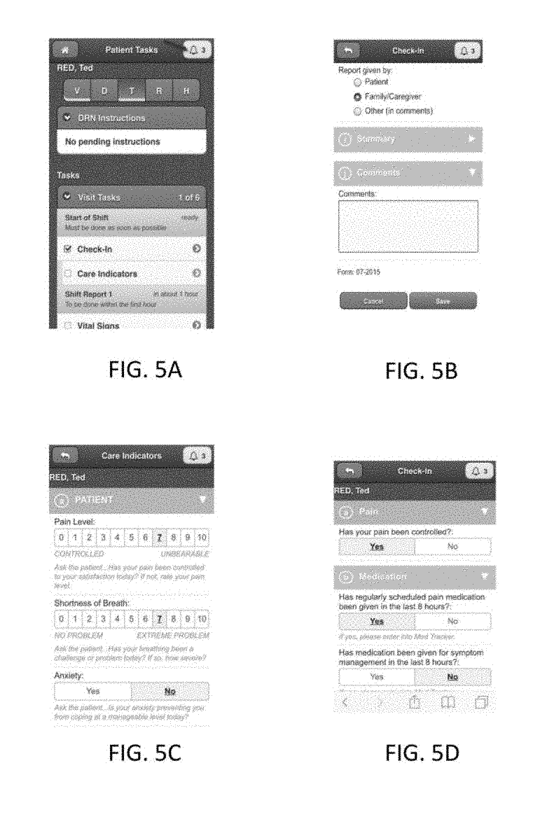

FIGS. 5A-5D present examples of the types of forms that a mobile user such as a technician may be presented with at the beginning of a shift.

Referring to FIG. 5A, an example of a form on a mobile device 8 is presented on the mobile device 8. The form is to be completed by a mobile user. In the example depicted in FIG. 5A, a first overview screen for a mobile device 8 is provided. The overview screen includes an overview of the tasks that the mobile user should perform during the shift. The overview screen also provides a window where a clinician can specifically instruct, through the directing clinician workstation 2, the mobile user to perform specific actions.

Referring now to FIG. 5B, a check-in screen is depicted on the mobile device 8. In this example, the check-in screen is presented once the check-in button in FIG. 5A is selected. The check-screen is configured to allow a mobile user to identify his or her role, provide a summary, and provide comments.

Referring now to FIG. 5C, a care indicator screen is depicted on the mobile device 8. In this example, the care indicator screen is presented once the care indicators button in FIG. 5A is selected. The care indicator screen allows a mobile user to enter care indicator information including, but not limited to, pain level, shortness of breath, anxiety, etc. In this example, instructions may be provided to ensure that the mobile user asks the correct questions to the patient. The instructions may be provided to the technician in real-time.

Referring now to FIG. 5D, a form is provided when a mobile user such as a technician asks questions to the patient and enters care indicator data. In this example form, the patient is asked various questions including, but not limited to, pain levels, pain medication administration, and symptom medication administration.

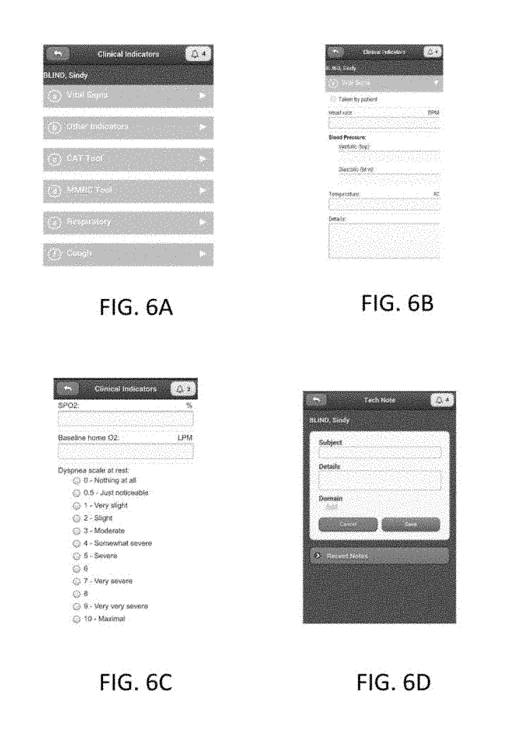

FIGS. 6A-6D present examples of the kinds of forms presented to the mobile user, such as a technician to collect and enter patient data.

Referring now to FIG. 6A, a clinical indicator summary screen is depicted on the mobile device 8. In this example, the clinical indicator screen is presented once a clinical indicator button (not shown) is pressed. This screen presents a series of buttons corresponding to various clinical indicators that a mobile user should enter. Pressing a button will bring up a corresponding form for the clinical indicator described on the button.

By way of example, FIG. 6B depicts a vital signs form. This vital signs form is brought up once the vital signs button in FIG. 6A is pressed. The vital signs form is configured to allow a mobile user to enter a patient's vital sign information. In this example, vital signs such as blood pressure, heart rate, temperature, and details can be recorded. Other vital sign indicators, such as, but not limited to, pallor, reflexes, and pupil sensitivity can be captured without departing from the scope of this disclosure.

Referring now to FIG. 6C, another clinical indicator form is depicted on the mobile device 8. In this example, the clinical indicator screen is configured to allow a mobile user to enter additional data regarding the patient's condition. In the example depicted in FIG. 6C, this indicator screen may be presented once a user presses the “other indicators” button on the mobile device 8. In this example, the form is configured to allow a user to enter data related to SPO2, baseline heme O2, and the Dyspnea scale at rest.

Referring now to FIG. 6D, a note form is depicted on the mobile device 8. In this example, the note form is configured to allow a user to enter additional notes for observations that may not have been captured in the forms. In the example depicted in FIG. 6D, this notes screen may be presented once the user presses a “note” button (not shown) on the mobile device 8.

Once these forms are completed by the mobile user, the data is then transmitted to the directing clinician workstation 2 by way of the server 6. The data is displayed for review by the clinician on a dashboard display on the directing clinician workstation 2. The data, as it is received by the server, may be displayed in real-time on the dashboard display of the clinician workstation.

The server 6 is also configured to store, analyze, and/or process the data and metadata submitted through the forms. The server 6 is also configured to capture, analyze, and/or process any data from the directing clinician workstation 2 or the observing clinician workstation 4. Data can include, but is not limited to, patient data and any data input in the forms. Metadata can include, but is not limited to, the time the data was entered, the IP address of the mobile device 8, the user of the mobile device, the time it takes to respond to a request from the mobile device, the length of any conversations between the user of the mobile device 8 and the clinician, and/or a recording of any conversations between the user of the mobile device 8 and the clinician. The metadata may also be associated with specific patient data/identifiable patient data so that a more complete patient record/clinical record/patient history, etc. can be compiled for that patient.

It should be noted that the data collected, the presentation of the form, and the layouts of the form can change depending on the data to be collected and the types of patients being cared for. Furthermore, the number of forms and order of forms can be changed without departing from the scope of this disclosure

The Directing Clinician Dashboard

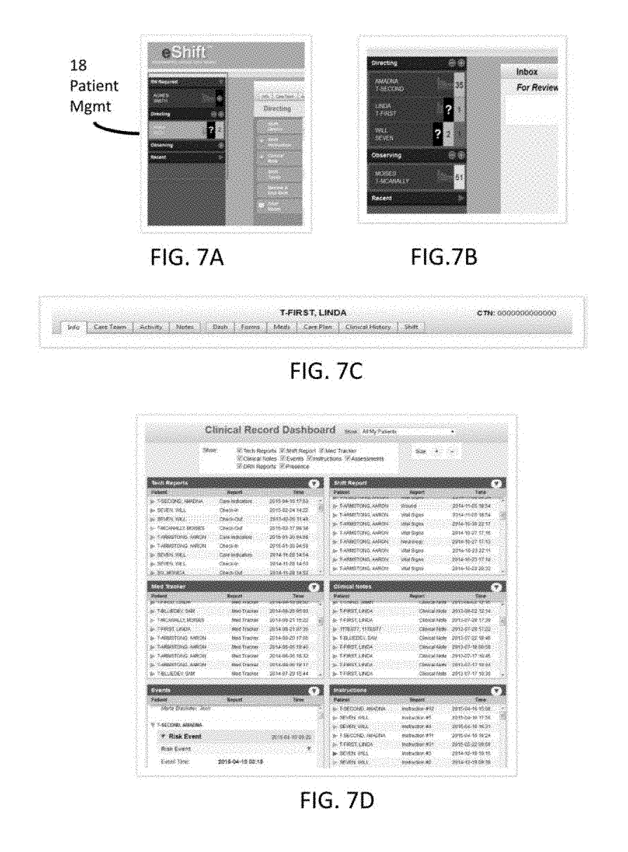

As can also be seen in FIGS. 7A-D, the dashboard for the monitoring computer displays not only the readings for the patient but also identifies the patient and the clinician monitoring the readings.

Also, part of the dashboard for the clinician is a window (not shown) that provides the user with a history of a particular patient's medical history and a history of the various readings taken of that patient's vital signs. This history of the patient's vital signs (from previous readings taken by mobile users) can allow the quickly determine, at a glance, whether the current readings are within acceptable parameters or not. By quickly comparing the current readings taken by the mobile user with the historical data, the clinician can determine whether further confirmatory readings are required or whether a dangerous condition is occurring. It should be noted that if the clinician determines that at least one reading is not within acceptable parameters, they may direct the mobile user to take more readings to determine if the previous readings were accurate. The direction to the mobile user to take more readings may happen in real-time. This may allow for time and cost efficient use of the technician's visit to the patient as it would not require a subsequent visit to the patient to take another reading or confirmatory reading.

Again, regarding the dashboard, the current readings or data entries for each patient can be provided side by side with the historical data for that same patient. A side-by-side comparison allows the clinician to quickly determine if the new data is within acceptable parameters of the historical data. Moreover, any outstanding instructions to the mobile user can be shown on the dashboard adjacent to the current readings.

Referring now to FIG. 7A, a portion of an example directing clinician dashboard is depicted. The directing clinician dashboard is configured to be displayed on the directing clinician workstation 2. The directing clinician dashboard may be implemented as one or more web pages on a web server. Alternately, the clinician dashboard may be implemented as an application to be run on a general purpose computer such as a personal computer. FIG. 7A depicts, at least in part, a patient management display and a part of a summary screen.

In this example, the patient management display 18 displays the patients currently being directed and or observed under the “Directing” and “Observing” headings. Recently managed patients may also be displayed under the “Recent” heading. The patient management display 18 is also configured to provide alerts to the clinician. Alerts may be raised for a variety of reasons that include, but are not limited to, when a mobile user requires assistance, when a registered clinician must provide authorization for the administration or performance of a certain task, emergency situations, or when a timer or alarm has expired. In the example depicted in FIG. 7A, the alert (as shown at the top of the patient management display) indicates that a registered nurse is required for the patient AGNES SMITH. The clinician may then click on the AGNES SMITH button to review and process the request.

FIG. 7A further depicts a portion of an information window. In this figure side tabs are depicted that allow the clinician to see the statuses of all their open patient records and quickly context-switch between patients, keeping all tools for patient direction within the patient record tabs. These detail pages also include, but are not limited to, shift details, shift instruction, clinical note, shift tasks, review & end shift, and chat room. It would be understood that other tabs to other detail pages could be added without departing from the scope of this disclosure.

Referring now to FIG. 7B, another view of a patient management display 18 is provided that depicts the patient management display 18 in a different state. In this state, the alert from FIG. 7A has been cleared. Furthermore, the number of patients being observed and directed has changed when compared to the patient management display 18 of FIG. 7A.

Referring now to FIG. 7C, another partial view of an information window is provided. In this figure tabs are depicted that allow the clinician to navigate from one function of the directing clinician workstation 2 to another. In this example, the tabs allow the clinician to quickly navigate between various information pages related to the patient LINDA FIRST. It will be appreciated that the tabs may be configured to allow the clinician to navigate to any page and/or function provided to the directing clinician workstation 2.

Referring now to FIG. 7D, a dashboard view of the directing clinician workstation 2 is provided. In this figure, the dashboard provides a summary of a number of recent entries of various aspects and/or functions of the directing clinician workstation. In this example, the dashboard provides a summary of recent “Tech Reports”, “Med Tracker”, “Shift Report”, “Clinical Notes”, “Events”, and “instructions”. It will be appreciated that the dashboard can be configured to display additional data, or different data from different aspects of the system without departing from the scope of this disclosure. For example, the dashboard may be configured to display the total number of alerts over a given period, or a chart depicting the number of patients that were administered medication at a given time of the day.

The Observing Clinician Dashboard

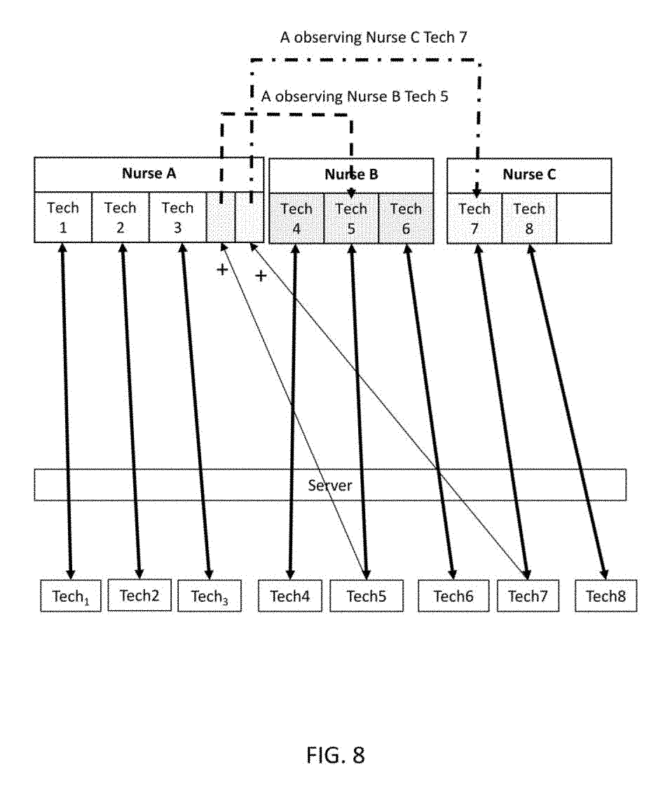

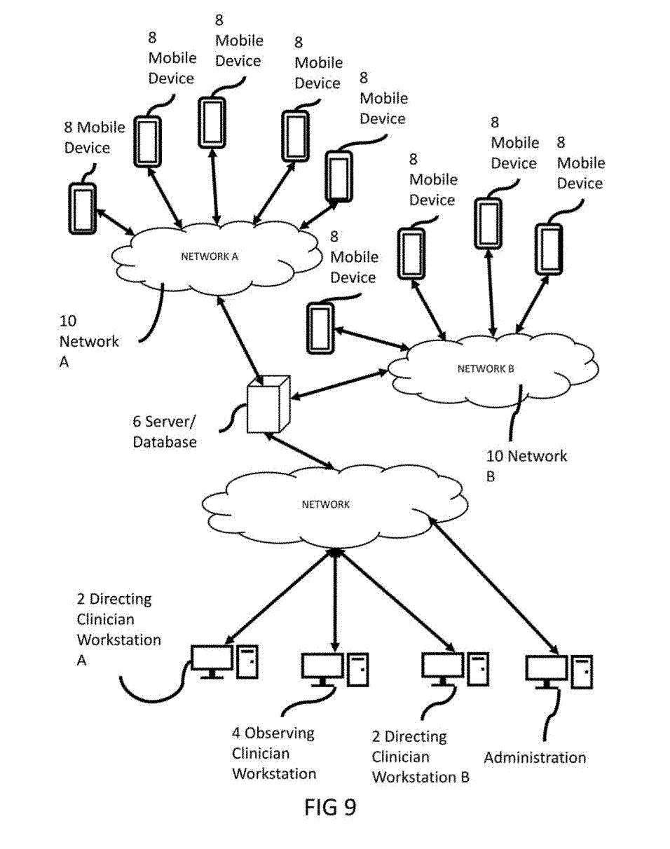

FIG. 8 illustrates the relationship wherein a clinician at a directing clinician workstation can also have observing permissions for one or more mobile users (eg., technicians) who are directed, supervised and managed by anther clinician at separate directing clinician workstation. The observing status provides the observer with a display for that specific technician and patient, but does not permit them to direct the technician, although the observing clinician does get access to the patient's chat room along with the directing clinician and the technician. In this manner, should an issue arise whereby the observing clinician needs to take control the technician, they can do so seamlessly. This functionality assists with work load balancing between the various directing clinician workstations. This work load balancing may occur in real-time.

The arrows in FIG. 8 illustrate how the communication between the directing clinician and the technician are bi-directional, but the data-flow between the observing clinician (for that technician) in only towards the clinician's observing section of the screen. The arrows indicating the flow of data from the technician to the observing clinician have a plus sign associated to indicate that the observing clinicians also have chat room privileges with the directing clinician and the technician.

FIG. 9 illustrates an embodiment of one configuration of the system emphasizing the situation depicted in FIG. 8. One skilled in the art would appreciate that there are not two separate internets in reality (ie., Network A and Network B) and that the separate “clouds” have been used to denote the plurality of mobile devices/mobile users/patients overseen by each directing clinician. For example, directing clinician A oversees the mobile device users associated with “network A” and directing clinician B oversees the mobile device users associated with “network B. The directing clinician A may also have observing status for some of the mobile users under the directing authority of directing clinician B and vice versa.

Two-Way Communication Between Clinician and Mobile User

One functionality provided by the system includes means by which, if the clinician sees anything amiss or anything that raises a concern, the clinician can initiate a two-way communication with the mobile user located with the patient. Similarly, if the mobile user sees anything that is of concern, the mobile user can initiate a two-way communication with the clinician through the directing clinician workstation 2.

The two-way communication referred to above may take various forms. An instruction and response type of communication (that is, a workflow based type) may be implemented where the clinician sends instructions to the mobile user of what to do. For each instruction, the mobile user then responds with confirmation that the instruction has been executed or that the instruction has not been executed, along with reasons why the instruction was not completed. The mobile user can also respond with a request for further clarification regarding the instruction. This two-way communication may occur in real-time.

This workflow-based communication is tracked on both a clinician's dashboard on the monitoring computer and on the mobile computing device. Each instruction is noted on the monitoring computer and on the mobile computing device. Each instruction can only be marked or treated as being done/executed by the clinician once they are satisfied with the response from the mobile user. Once an instruction has been marked as being executed by the registered medical professional, the instruction is similarly marked on the mobile user's mobile computing device. All of the instructions and the mobile user's responses to the instructions are stored in the database and are associated with the particular patient to whom it applies.

Another form of a two-way communication may be through well-known encrypted chat/text communications protocols where a free-flowing conversation between the registered medical personnel and the mobile user can develop. This communications channel allows for low patient impact and silent conversations between the mobile user and the registered medical professional. These chat/text communications may be logged in the database and may be manually associated with a specific patient, though in some implementations this may not be required.

Multi-Party Patient Chat Room

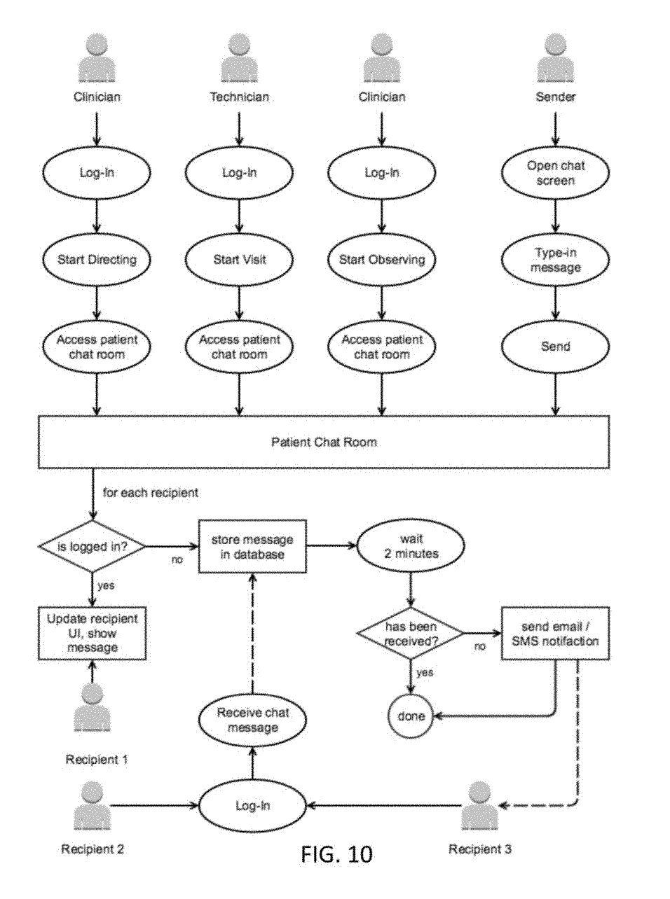

FIG. 10 illustrates the workflow for a multi-party chat room, which is available to individuals who have permission to access a patient record. These individuals can be the directing clinician, the mobile user, all observing and consulting clinicians. In some embodiments, it may be appropriate to allow other individuals in roles to be able to access the patient record and participate in the chat room.

The patient chat room feature provides a patient-specific messaging functionality within a patient record. The patient chat room allows instant chat-like communication between participants who are actively providing care to a specific patient. A patient chat room is unique and separate from other patient chat rooms (i.e., each patient has their own chat room uniquely associated with their patient record) and records the chat conversation (transcribes the conversation) between the active participants on the patient.

Once a patient record is created in this system, a chat room is available, such that any individuals with permissions to access the patient record can leave messages in the chat room for themselves and/or others. When the patient chat room is available, observing and consulting clinicians can also access the patient chat room.

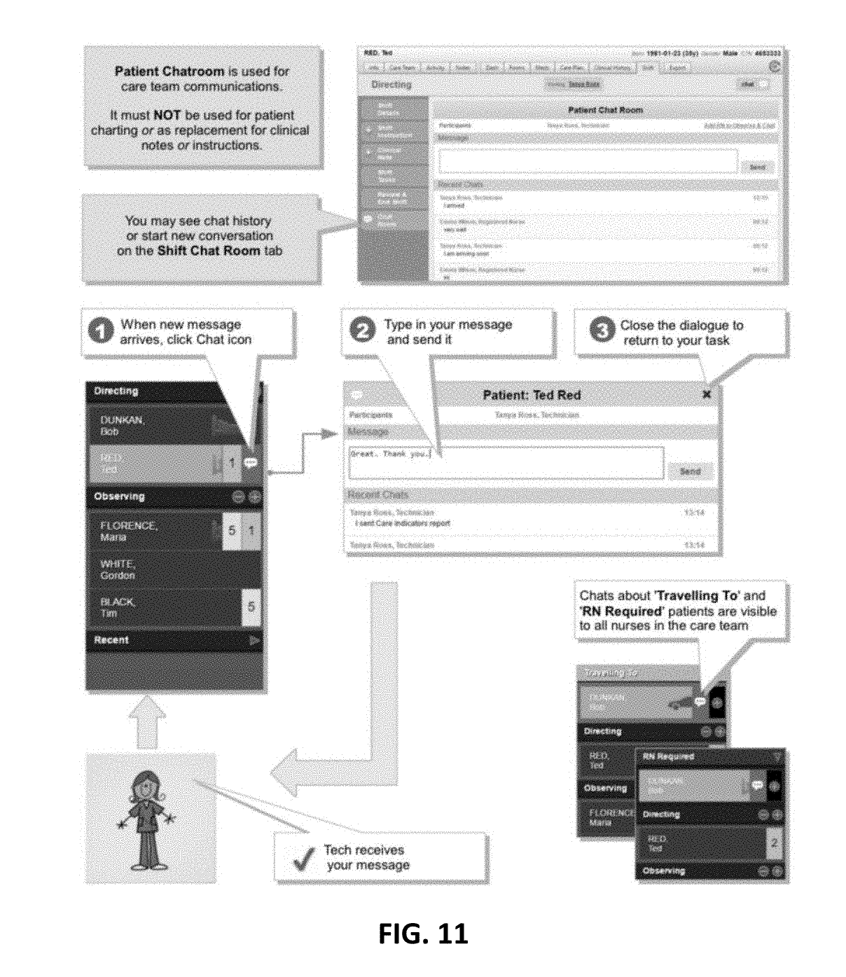

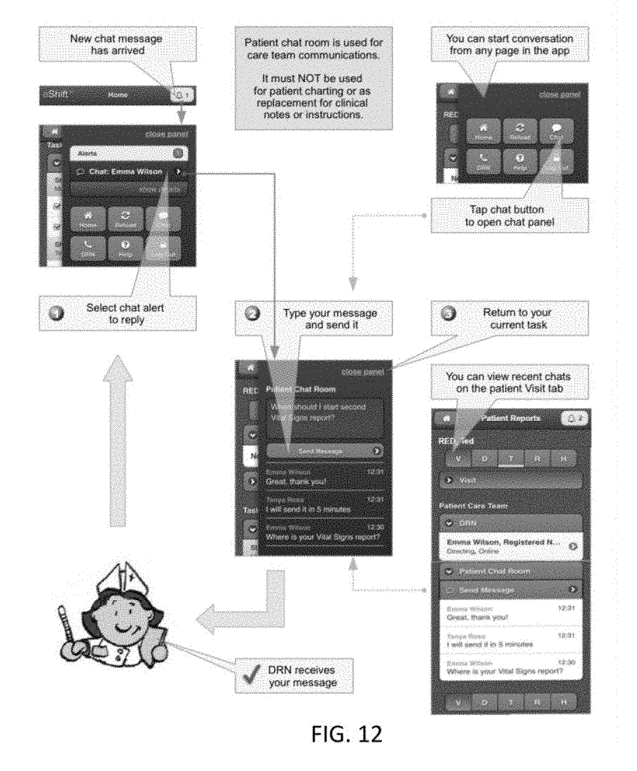

When viewing the active patient chat room screen, all available participant names are displayed on screen. To send a chat message, the user types their message and sends it. When the chat message arrives on the server, the server sends new chat notifications to all current participants on this patient. This is a blast-broadcast notification system. The chat room is closed when directing clinician stops directing in the patient record. FIG. 11 provides exemplary screen shots and instructions for the chat room functionality that could appear on the screen of any users that are using the web functionality. FIG. 12 provides exemplary screen shots and instructions for the chat room functionality that could appear on the screen of any users that are using the mobile functionality.

The Care Team

In one embodiment, the Care Team comprises individuals organized by roles, who have clinical access to a patient record. Each role generally has its own unique set of permissions and functionalities that are relevant and appropriate to their responsibilities of caring for a patient. Different patients can have different roles involved in their care team. Different patients who have the same roles involved in their care can have different individuals in these roles.

In one embodiment, the members of the Care Team can be assigned by name (for example Drs. A, B and D, nurses X, Y, Z & P, technicians #1, 2, 3) and whoever is available at a point in time would be considered to be the individual currently available for that patient and other members of the team. In one embodiment, the roles of the Care Team can be assigned to a patient and the system will keep track and display who is currently active in that role.

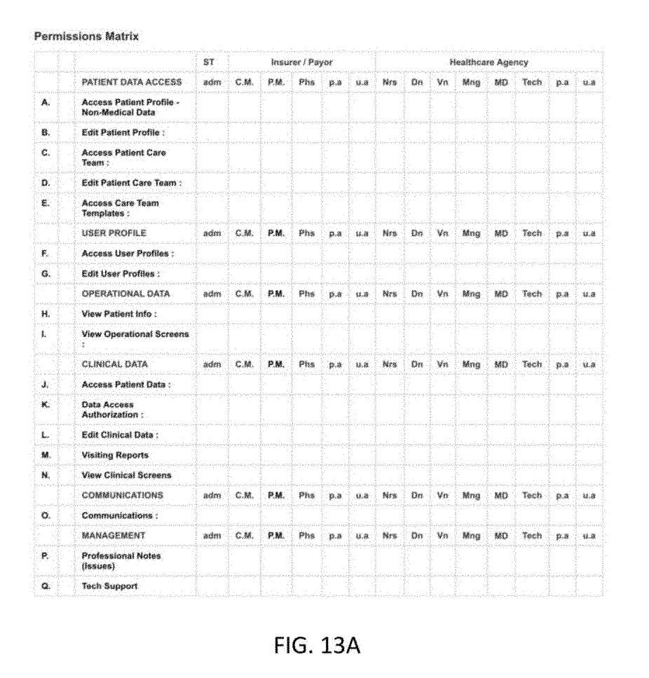

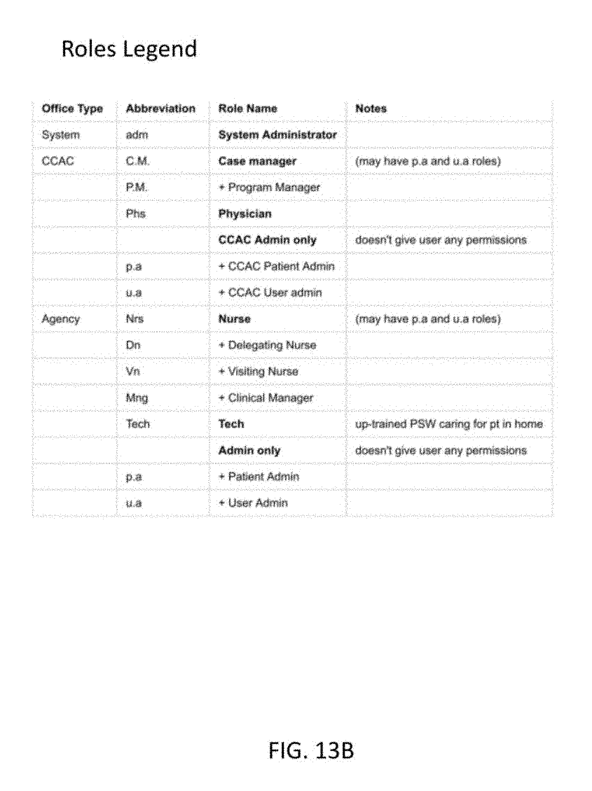

FIG. 13A illustrates some exemplary roles and possible permissions/functionalities that could be associated with the roles. FIG. 13B describes the legend of role names presented in FIG. 13A, which also illustrates some of the possible general roles that may be included in a Care Team for a patient. Note that CCAC (Community Care Access Center) is included as an example of an Insurer/Payor.

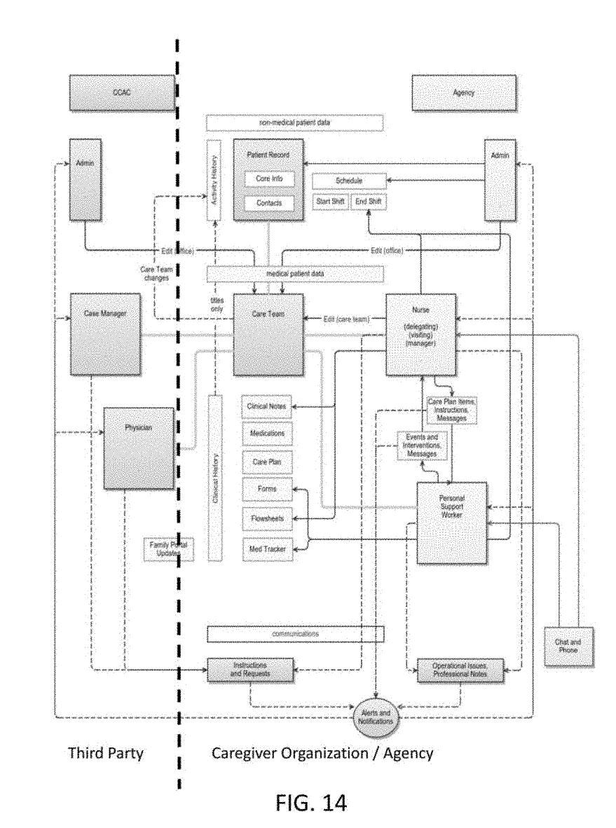

FIG. 14 illustrates one embodiment of a system configuration wherein the Care Team comprises members from a third-party organization, who are essentially observers watching the performance of the Caregiver Organization/Agency, which comprises the directing, observing and consulting clinicians, mobile users, administrators, etc., who are using the system, according to one exemplary workflow.

Managing the communication includes, but is not limited to, storing and retrieving data entered into the system by any party; initiating, facilitating, and logging voice, chat, email, video, and/or blog communications between parties using the system; generating alerts and/or notifications and directing the alerts and/or notifications to the appropriate party; and/or generating, retrieving, and storing metadata of the data entered into the system and any communications initiated or facilitated by the system.

In this example, the system retrieves and stores data related to, among other things, technician schedules and their associated start and end shift times, nurse schedules and their associated start and end shift times, admin schedules and their associated start and end shift times, the patient record, the patient's medical data, the patient's non-medical data, clinical notes, medications, care plans, forms, flowsheets, and medical trackers. The system is also configured to allow nurses, agency administrators, and/or personal support workers to access and/or modify the data stored in the system's datastore. Access to the data stored in the system's datastore may occur in real-time.

In this example metadata based on the stored data is also captured by the system and stored in the system's datastore. This metadata can be used to analyze and report on, among other things: the attendance of a nurse, admin, or personal care worker; a patient's clinical history; an activity history (and associated metadata such as date of administration, time taken to perform an activity, time to report an activity, etc) of each activity performed.

The system may also be configured store contact information of each party and/or person that has access to the system's datastore. This data would also be stored in the system's datastore.

In this example, the system may also be configured to send alerts, messages, and notifications to any party and/or person that has access to the system. For instance, the system may act as a conduit for transmitting alerts, messages, and/or notifications between physicians (on the third-party side) to the nurse, personal support worker, and/or admin on the agency side. In some examples, the care team may store the alerts, messages, and/or notifications, or a record of the above information, in the care team datastore. In other examples, a metadata of the alerts, messages, and/or notifications may also be stored in the care team datastore.

In the example provided in FIG. 14, the system may be implemented on one or more servers 6. The servers 6 may be deployed remotely relative to the patient facility, or locally at the patient facility. Alternately the servers 6 may be implemented in the cloud.

The system may include one or more datastores for storing data. For example, the care team may store the patient data, medical data, and all related metadata in a single datastore on a server 6. Alternately, the care team may store data across several datastores. In one example, complete copies of the datastore may be replicated to another server 6 in order to allow for a failsafe should the first datastore become corrupted. In another example, a secondary datastore containing a subset of the data in the care team's datastore may be included, for example, where the data is anonymized. This secondary datastore would be used, for example, to limit access to only the data required for third-parties to perform their task. This might mitigate, at least in part, any liability related to data privacy regulations when allowing a third-party to access medical data.

In some examples, the data in the system's datastore may be encrypted, at least in part. The encryption might mitigate, at least in part, any liability related to data privacy regulations should the system become compromised (e.g., accessed by an unauthorized party, leaked to the public, etc).

Referring again to FIG. 14, in the system provided in FIG. 14, parties in the Care Team (e.g., the nurse or clinician, agency admin, and/or the technician) can communicate with each other through the system. The system may also manage the data flow between parties in the Care Team. The nurse may provide instructions, review data input by the technician, review and respond to any events or interventions or messages from the technician, and so on. Likewise, a technician can input data into forms, take notes on the patient, send alerts to the nurse. Both the nurse and the technician may also initiate communications with the other party. In this example, the both the nurse and the technician are capable of initiating voice or chat messaging between each other. Finally, the agency admin may schedule a technicians' or nurse's shift, review their start and end times, and administer any aspect of the system from a Care Team perspective. This can include reviewing data, metadata, and any communications between a technician, nurse, and/or agency admin.

It will be appreciated that, in some example embodiments, the system may contain several layers of licensed medical professionals, and that each layer may be capable of initiating communication with any other layer. For instance, in an example embodiment the system may also have a licensed physician, administrator, etc. who would occupy a higher layer in the hierarchy. The licensed physician (or higher layered individual) may be tasked with observing, managing, and/or directing any number of regulated or non-regulated personnel including, but not limited to, registered nurses, nursing assistants, and/or technicians. In some example embodiments, the physician (or higher layered individual) may be logged into a directing clinician workstation 2. In other example embodiments, the physician (or higher layered individual) may be logged into an observing workstation. In yet another embodiment, the physician (or higher layered individual) may be logged into an Administration workstation.

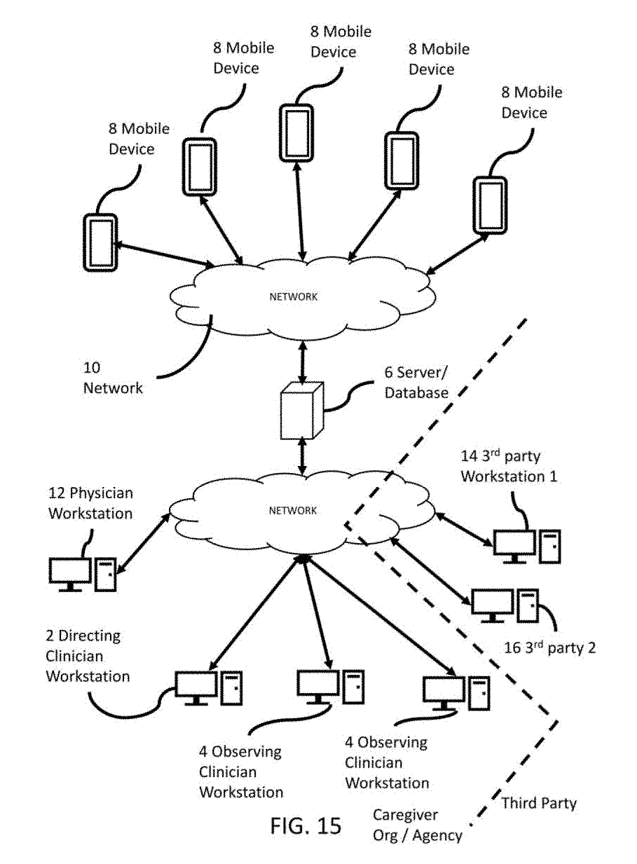

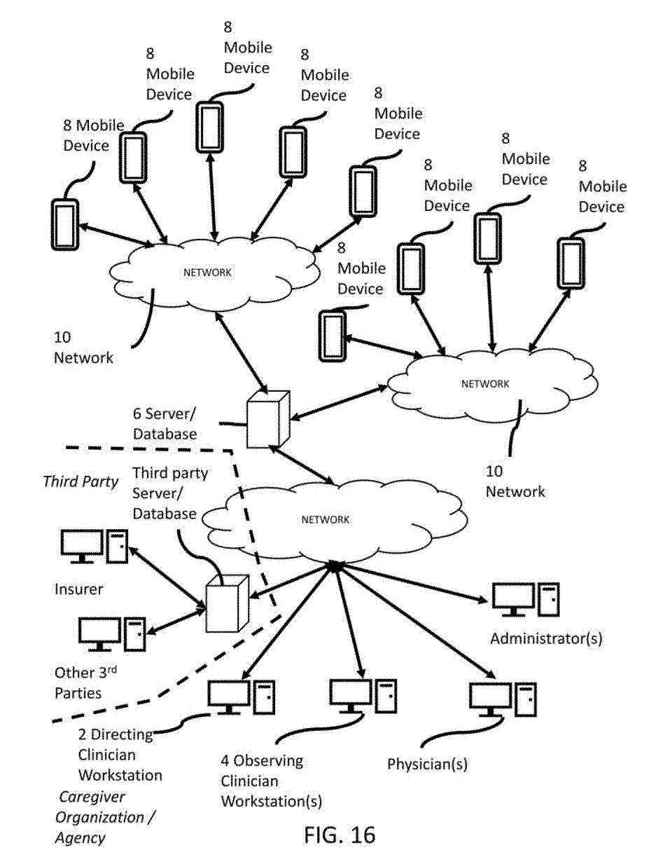

FIG. 15 and FIG. 16 illustrate different embodiments comprising configurations that would support the Care Team example presented in FIG. 14. FIG. 15, shows a configuration wherein all the parties access the server/database which is hosted in a secure datacenter. FIG. 16 illustrates a configuration wherein a third-party would have their own data warehouse wherein anonymized data, for example, would be extracted from the Clinical database for further processing. such as report generation and utilization analysis.

Parties External to the Care Team

In the embodiment described in FIG. 14, third parties (eg. CCAC) may also communicate with the system and the parties in the Care Team. The system may also manage the data flow between the parties in the care team and the third parties. In some examples. the third party may only be permitted access to a subset of the data in the care team's datastore (e.g., anonymized). This may be useful, for example, in protecting the privacy of a patient.

In the embodiment provided in FIG. 14, the third party may be allowed to observe, provide instructions, and/or make requests to the clinician (e.g., nurse) or the technician through the system. For example, a third party physician that is outside of the care team/agency may modify a drug dosage for a patient, which would then be communicated to the clinician, technician, or both, through the system. Similarly, a case manager may approve a treatment plan proposed by a physician and stored on the core team's datastore. This approved plan might then be communicated to the clinician, technician, or both through the system.

A third party might include, but is not limited to, an insurer, government body, oversight committee, hospital network, public health researchers, or any group that may be interested in patient data, including anonymized patient data.

Referring now to FIG. 15, yet another representation of the system is depicted. In this example, all of the terminals are located remotely from the server 6. For instance, in this example the server 6 may be implemented in the cloud (on an AMAZON EC2 instance, for example) so that any terminals accessing the server 6 would do so remotely.

Referring now to FIG. 16, an alternate representation of the system is depicted. In some examples, the server 6 may include a separate database for use exclusively by the third-party. In this case, as depicted in FIG. 14, a subset of the data may be replicated from the caregiver database (i.e., the main database) on the server to a third-party server or database. The third party will then only be able to access data stored on the third party server/database. This may mitigate the risk of sensitive patient data being inadvertently exposed to unauthorized parties.

The System Manages the Dataflow Communications Between (the Parties)

Referring again to FIG. 14, in this example three functional groups are identified. The three functional groups are the system, the caregiver organization/agency, and the third party payor/insurers (CCAC).

The system is generally responsible for managing the communications and data flow between the parties of the caregiver organization/agency (i.e., the clinicians and the mobile device users) and the communications between the agency and the third-party.

Two-way communications can then be initiated between any of the members of the care team. This communication may then be stored, logged, and/or tracked in the system. This communication may also be associated with the patient record.

How the External Parties Communicate with the System

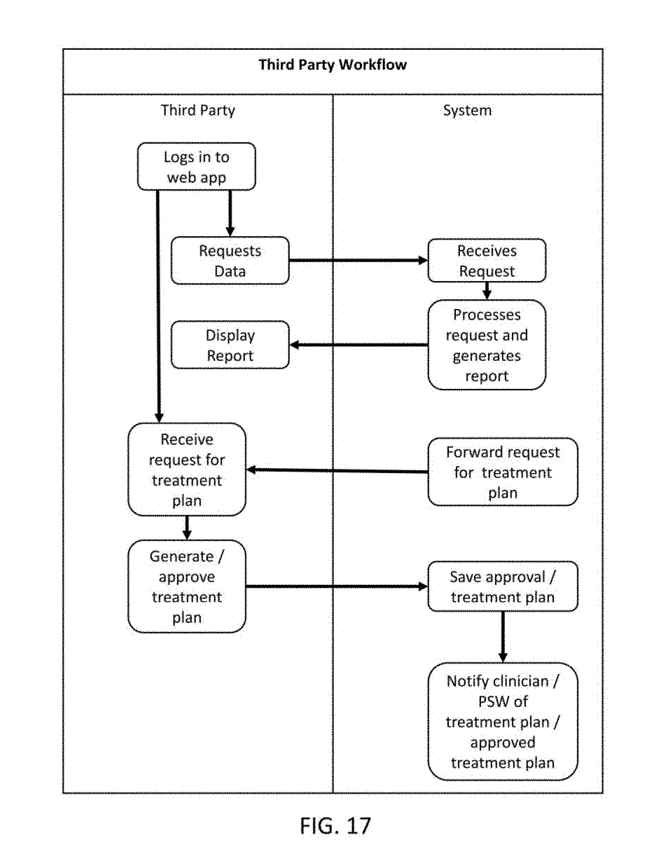

Referring now to FIG. 17, an example workflows between a third party and the care team system is provided. In the first workflow, a third party (such as a representative of an insurance company, etc) logs into a third party workstation. In this example, the third party may wish to view all of the activities performed over a 24 hour period, and makes the corresponding request to the system. The system receives the request and then processes and generates the report accordingly. In this example, the system may anonymize data that is stored in the care team's datastore, generate a report, and display the report on the third party's workstation. In another example, the care team's system may first replicate the data to a second data store (as was discussed in FIG. 17). This second data store may contain a subset of the data (that may be anonymized or otherwise clear of identifiable patient information) in the care team's primary data store. This may mitigate, at least in part, liability associated with a breach of patient data should the secondary datastore become compromised. The reports may then be generated using the care team's secondary datastore, and then presented to the third party.

In the workflow depicted in FIG. 17, once the third party logs in the third party may receive a request for a treatment plan from the system. In this workflow, a nurse, clinician, consultant, or technician will have requested, at an earlier stage, a treatment plan. In some examples, the third party may have been notified of a pending treatment plan request over email or SMS. The third party (in this case, a licensed physician) generates and/or approves the treatment plan. This is then transmitted to the system, which saves (into the datastore) and/or processes the approval/treatment plan. The care team system then notifies the clinician/technician of the approval/treatment plan.

It will be appreciated that metadata, data incidental to the workflow, or non-medical data may also be captured at every step of the workflows previously described. This metadata can be stored in the system's datastore or a secondary datastore. Analysis may be performed on the metadata. This analysis may be used, for example, to determine the efficiency of each of the clinicians, personal support workers, etc. The metadata analysis may also be used to determine the average response time, number of requests per hour, and any other information that may be of interest to a user of the system. A skilled person would understand that other metadata may be collected without departing from the scope of this disclosure. Furthermore, a skilled person would understand that the steps in the workflows are intended to describe, at a high level, the workflow of the system. The actual implemented workflows may vary from the workflows as described, and that these variations within the scope of this disclosure.

The Consult Functionality

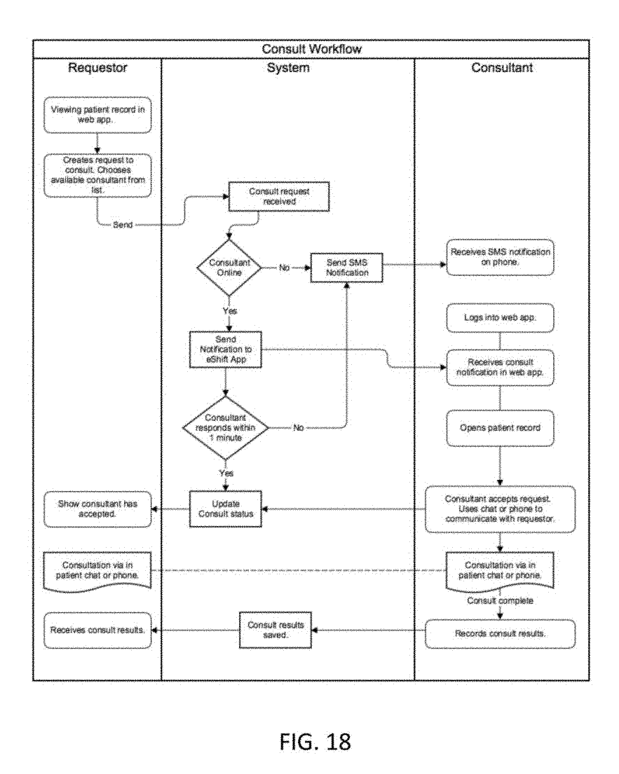

Referring now to FIG. 18, an exemplary workflow for a member of a Care Team (or caregiver organization, or agency) seeking a consultation with one or more other members of a patient's Care Team using the system is depicted. In this workflow, a requestor, for example a visiting nurse located in a patient's home, a non-hospital location, a care facility of a patient, or other remote location, views a patient's electronic medical record in the web application, which is displayed on the mobile device 8. In this example, the visiting nurse may create a consultation request in the patient's electronic medical record on the mobile device 8, to communicate with another member of the Care Team such as a nurse, physician or other role located remotely from the patient. The nurse may have the option to select one or more clinicians or other member of the team to consult with (i.e. “consultants”) from a list that is provided on the mobile device 8 indicating the members on the Care Team of the patient. Once the visiting nurse selects the consultant and presses the send button, a message is sent from the mobile device 8 to the system (in this case, the server 6). The consultation may occur in real-time.

Once the server 6 receives the consultation request, the system is configured to process the request from the mobile device 8. In this case, the system determines whether the consultant is logged into the web application. If the consultant is not logged into the web application an SMS or email message is sent to the consultant to alert them that a visiting nurse has made a request for a consultation. Similarly, if the consultant is logged in to the web application but does not respond to the request within a set amount of time (one (1) minute in this example) an SMS or email message is sent to the consultant to alert them that a visiting nurse has made a request for a consultation.

If the consultant is already logged in, the consult notification is received on the web application alerting the consultant that a consultation request is pending. The consult notification may then be displayed on the directing clinician workstation 2. The consultant may proceed to accept or reject the request. In the case where the consultant is not logged in but logs in once a request is received from the visiting nurse, when the consultant logs into the web application a consult notification is received on the web application, and the consult notification may then be displayed on the directing clinician workstation 2. The consult notification may contain additional data relevant to the consultation request that may not have been contained in the email or SMS message. The additional relevant data may include urgency, purpose for consult request, patient specific data, and other information necessary that is live or in real-time for the consultant to quickly decide to accept or reject the consult or actions to take during the consult.

In some examples metadata related to the time between the initial request and the time a consultant first logs into the system may be captured (i.e., response time). Other metadata, such as the amount of time a consultant reviews the patient record, the length (e.g., number of words) of the consult notification, the time of day of the consult, the number of consults for the specific patient, number of notifications sent, whether an email or SMS notification had to be sent, etc. may be captured and stored in the patient's record.

The consult notification links to the patient's electronic medical record, which allows the consultant to access and quickly review the patient record. The patient's electronic medical record is continually updated by the system, in that the information contained therein is live or real-time to ensure that the consultant and the visiting nurse may make more accurate and quick decisions regarding the patient. Once the consultant accepts the consultation request, the patient's electronic medical records opens for the consultant's review. The consultant may review the patient's electronic medical record prior to accepting the consultant request or otherwise communicating with the requestor (i.e., the visiting nurse). Once the consultation is accepted, the system records, in the patient's electronic medical record, the consultant's response, sends the response to the visiting nurse, and may propagate the status change (i.e., that they accepted to become a consultant) to the requestor (or visiting nurse) by updating, for instance, the state on the mobile device 8.

If the consultant rejects the consultation request, the system records the response and sends the response to the visiting nurse. The visiting nurse may cancel the request or initiate additional consultation request. The consultation request will remain open in the system until the request is responded to, canceled by the visiting nurse, or the request is closed by system. A request may be closed by the system if the visiting nurse ends their shift and leaves the patients home. Having the system close the consultation request maintains the relevancy of the consultation request queue, and prevents time wasted by consultants on reviewing no longer necessary or expired consultation request.

Accepting the request may allow the consultant to initiate a video, voice, or text communication with the requestor. In some cases, the communication may be facilitated through a direct connection from the consultant to the requestor (e.g., a phone call, etc). In other embodiments, the system may include a communications system that connects the consultant to the requestor. For instance, a chat (e.g., the Multi-Party Patient Chat Room), voice communication, or video communication application may be implemented on the system so that a consultant and a requestor communicate with each other through infrastructure provided by the system. This allows the system to capture and record any communications between the requestor and the consultant within a patient's electronic medical record so that a more detailed and complete patient record may be compiled.

Once the communications between the requestor (i.e., visiting nurse) and the consultant is complete, the system may present the consultant with a form to fill out, the consultant records the results of the consult and submits it to the system. The results of the consult (i.e., the data) and any associated metadata is then stored on the system's datastore within the patient's electronic medical record. This data may also, in some instances, be associated with the patient's electronic medical record so that a more detailed patient history can be compiled. In some example embodiments, the requestor (i.e., the personal care worker) may also receive the entirety or a subset of the information recorded by the consultant.

Example methods and an example system are provided below:

Method 1: A method of consulting with a plurality of patient electronic medical records, and one or more members of a patient's Care Team connected electronically through a system to one another, the method comprising the steps of:
a. a user member of a patient's Care Team who is logged into the system entering a patient's electronic medical record and viewing members of the patient's Care Team that are possibly available for consultation at a specific time period;
b. a user member selecting one or more members of the patient's Care Team for consultation at a specific time period and sending one or more requests for a consultation regarding the patient to the system, thereby designating the one or more authorized members to be potential consultants;
c. receiving the one or more requests in the system;
d. for each potential consultant requested, the system determining whether the potential consultant is online;
   if the potential consultant is online,
      1. sending a notification through the system;
      2. receiving notification of the consultation request on the potential consultant's device;
      3. displaying the consultation request on the potential consultant's device;
      4. the system determining whether the potential consultant responds to the notification within an (“allowable response time”);
         a. if the potential consultant does not respond within an (“allowable response time”), the system sending a (SMS or email) notification of the request to the phone or message device of the potential consultant
   if a potential consultant is offline,
      1. the system sending a (SMS or email) notification of the request to the phone or message device of the potential consultant;
      2. the potential consultant logging onto the system;
e. the potential consultant sending an acceptance or denial response of the requested consultation to the system;
f. the system opening the patient's electronic medical record for the potential consultant's review;
g. the system recording the response of the potential consultant in the patient electronic record;
h. the system sending notification of the response of the potential consultant to the user member;
i. the user member and the one or more consultants conducting the consultation (using the system chat room functionality or by phone);
   if by chat room functionality—the system records the consultation and the timing in the patient record
j. when the consultation is completed, the system presenting a form to the one or more consultants to report the results of the consultation;
k. the one or more consultants sending a completed consultation report to the system; and
l. the system saving the completed consultation report in the patient electronic medical record.

Method 2: A method of consulting with a plurality of patient electronic medical records and one or more members of a patient's Care Team connected electronically through a system to one another, the method comprising the steps of:
a. a user member of a patient's Care Team who is logged into the system entering a patient's electronic health record and viewing members of the patient's Care Team that are possibly available for consultation at a specific time period;
b. a user member selecting one or more members of the patient's Care Team for consultation at a specific time period and sending one or more requests for a consultation regarding the patient to the system, thereby designating the one or more authorized members to be potential consultants;
c. receiving the one or more requests in the system;
d. for each potential consultant requested, the system determining whether the potential consultant is online;
   if the potential consultant is online,
      1. sending a notification through the system;
      2. receiving notification of the consultation request on the potential consultant's device;
      3. displaying the notification on the potential consultant's device;
      4. the system determining whether the potential consultant responds to the notification within an (“allowable response time”);
         a. if the potential consultant does not respond within an (“allowable response time”), the system sending a (SMS or email) notification of the request to the phone or message device of the potential consultant
   if a potential consultant is offline,
      1. the system sending a (SMS or email) notification of the request to the phone or message device of the potential consultant;
e. the potential consultant logging onto the system;
f. the potential consultant opening the patient's electronic health record;
g. the potential consultant sending an acceptance or denial response of the requested consultation to the system;
h. the system recording the response of the potential consultant in the patient electronic record;
   if an acceptance response, the system updating the availability status of the of the potential consultant to an occupied consultant for the requested time period (therefore not available for other requests);
   if a denial response; the system maintains the status of the potential consultant;
i. the system sending notification of the response of the potential consultant to the user member;
j. the user member and the one or more consultants conducting the consultation (using the system chat room functionality or by phone);
   if by chat room functionality—the system records the consultation and the timing in the patient record
k. when the consultation is completed, the system presenting a form to the one or more consultants to report the results of the consultation;
l. the one or more consultants sending a completed consultation report to the system; and
m. the system saving the completed consultation report in the patient electronic medical record.

A system for providing health care to a patient located outside of a hospital comprising:
a. one or more mobile computing devices configured to connect wirelessly to the internet, each of the devices having a graphic user interface comprising one or more data entry elements and configured to display graphics that direct a worker to collect and enter into the device specific patient data and further configured to transmit the specific patient data to a main computer, receive instructions from a monitoring computer, and mark an instruction as completed;
b. a main computer configured to transmit a plurality of menus, forms and interfaces to each mobile computing device, receive the data from each mobile computing device, store the data in a database and transmit the data to a monitoring computer, establish a two-way communication between the monitoring computer and one or more of the mobile computing devices, assign and reassign mobile computing devices to a monitoring computer;
c. one or more databases configured to store and retrieve patient data; and
d. a monitoring computer configured to receive and display patient data inputted into the mobile computing devices by one or more workers, transmit instructions through a two-way communication interface to the mobile computing devices,
e. one or more computing devices (other Care Team members) configured to receive and display the electronic health record of a patient (stored in the database)
wherein the main computer is configured to execute the method of claim 1.
A system for enabling a clinician to monitor the health status of a patient situated in a non-hospital location and attended by a non-licensed worker, the system configured to execute the method of example 1 or 2.

Implementing the embodiments of the system and method described above enables a visiting nurse, technician, or non-licensed medical professional to work independently, in a patient's home, care facility or non-hospital location, while having immediate and real-time access to authorized clinicians, such as registered nurses, physicians or other licensed medical professionals who are updated and familiar with the patient. Additionally, the clinicians or consultants will have real-time patient specific data through the patient's electronic medical record thereby allowing the clinicians or consultants to direct the visiting nurse regarding the patient's care. The immediate access to documentation and patient specific data contained within the patient's electronic medical record creates an additional layer of support and level of care for the patient that would not otherwise be available in traditional paper and non real-time systems. Consultants and visiting nurses may rely on the information received, directions provided, actions taken, and patient status. All data and metadata sent through the system concerning a patient is recorded in the patient's electronic record creating a complete, real-time and continuous patient record that is auditable and accountable.

It will be understood that the same workflow may be applied to different parties of the system without departing from the scope of this disclosure. For instance, a similar workflow may be applied between a third-party (e.g., a physician) and the consultant (i.e., a clinician). A similar workflow might also be applied between a third-party (e.g., a physician) and the requestor (e.g., a technician). A similar workflow might also be applied between parties in the same organization (e.g., from one clinician to another clinician in the agency, or from one physician to an insurer from the third-party). This would allow for capturing data and facilitating communications between all parties that use the system.

This written description uses examples to disclose the invention, including the best mode, and also to enable any person skilled in the art to make and use the invention. The patentable scope of the invention is defined by the claims, and may include other examples that occur to those skilled in the art. Such other examples are within the scope of the claims if they have structural elements that do not differ from the literal language of the claims, or if they include equivalent structural elements with insubstantial differences from the literal language of the claims.

It may be appreciated that the assemblies and modules described above may be connected with each other as required to perform desired functions and tasks within the scope of persons of skill in the art to make such combinations and permutations without having to describe each and every one in explicit terms. There is no particular assembly or component that may be superior to any of the equivalents available to the person skilled in the art. There is no particular mode of practicing the disclosed subject matter that is superior to others, so long as the functions may be performed. It is believed that all the crucial aspects of the disclosed subject matter have been provided in this document. It is understood that the scope of the present invention is limited to the scope provided by the independent claim(s), and it is also understood that the scope of the present invention is not limited to: (i) the dependent claims, (ii) the detailed description of the non-limiting embodiments, (iii) the summary, (iv) the abstract, and/or (v) the description provided outside of this document (that is, outside of the instant application as filed, as prosecuted, and/or as granted). It is understood, for this document, that the phrase “includes” is equivalent to the word “comprising.” The foregoing has outlined the non-limiting embodiments (examples). The description is made for particular non-limiting embodiments (examples). It is understood that the non-limiting embodiments are merely illustrative as examples.

What is claimed is:

1. A method of consulting with a patient's electronic medical record, and one or more members of a patient's Care Team connected electronically through a system to one another, the method comprising the steps of:
a. a user member of a patient's Care Team who is logged into the system entering a patient's electronic medical record and viewing members of the patient's Care Team that are possibly available for consultation at a specific time period;
b. the user member selecting one or more members of the patient's Care Team for consultation at a specific time period and sending one or more consultation requests for a consultation regarding the patient to the system, thereby designating the one or more authorized members to be potential consultants;
c. receiving the one or more consultation requests in the system;
d. for each potential consultant requested, the system determining whether the potential consultant is online;
i. if the potential consultant is online,
1. sending a notification of the consultation request through the system to the potential consultant's device;
2. receiving notification of the consultation request on the potential consultant's device;
3. displaying the consultation request on the potential consultant's device;
4. the system determining whether the potential consultant responds to the notification within a predetermined response time;
a. if the potential consultant does not respond within the predetermined response time, the system sending a notification of the consultation request to the potential consultant;
ii. if a potential consultant is offline,
1. the system sending a notification of the consultation request to the potential consultant;
2. the potential consultant logging onto the system;
e. the potential consultant sending an acceptance or denial response of the requested consultation to the system;
f. the system opening the patient's electronic medical record and displaying the consultation request data to the potential consultant;
g. the system recording the response of the potential consultant in the patient electronic record;
h. the system sending notification of the response of the potential consultant to the user member; and
i. the system recording and storing the consultation request data and metadata in the patient's electronic medical record.

2. The method of claim 1 further comprising, the user member and the one or more consultants conducting the consultation using the system infrastructure, the system recording the consultation, the consultation data and metadata in the patient's electronic medical record.

3. The method of claim 1 wherein,
a. if an acceptance response, the system updating the availability status of the of the potential consultant to an occupied consultant for the requested time period so that the potential consultant is not available for other requests; and
b. if a denial response, the system maintains the status of the potential consultant.

4. The method of claim 2 wherein,
a. when the consultation is completed, the system presenting a form to the one or more consultants to report the results of the consultation;
b. the one or more consultants sending a completed consultation report to the system; and
c. the system saving the completed consultation report in the patient's electronic medical record.

5. The method of claim 2, wherein the system infrastructure for conducting the consultation is any one of a chat room application, a voice communication application, or a video communication application.

6. The method of claim 2, wherein the consultation data and metadata is any one or a combination of the consultation date, consultation time, response time, IP addresses of mobile devices, time of acceptance or denial, consultation duration, method of consultation, and number of consults.

7. The method of claim 1, where the consultation request data and metadata is any one or a combination of the user member, the potential consultants, the consultation request date, the consultation request time, notification date and times, number of notifications sent, notification method, response times, IP addresses of mobile devices, response, purpose of consultation, or patient specific data.

8. The method of claim 1 wherein:
a. The notification of step (d)(ii) is at least one of a SMS message sent to the phone number of the potential consultant, and an email message sent to the email address of the potential consultant.

9. The method of claim 1 wherein:
a. The notification step of step (i)(4)(a) is at least one of a SMS message sent to the phone number of the potential consultant, and an email message sent to the email address of the potential consultant.

10. The method of claim 1, wherein the potential consultant is a physician, registered nurse, or licensed medical professional.

11. The method of claim 1, wherein the user member is a personal support worker, technician, or unlicensed medical professional.

12. A system for providing health care to a patient located outside of a hospital comprising:
a. one or more mobile computing devices having a graphic user interface comprising one or more data entry elements, the mobile computing devices configured to:
i. connect to the internet,
ii. display graphics that direct a user member to collect and enter specific patient data,
iii. transmit the specific patient data to a main computer,
iv. receive instructions from a monitoring computer, and
v. mark an instruction as completed;
b. a main computer configured to:
i. transmit a plurality of menus, forms and interfaces to each mobile computing device,
ii. receive the data from each mobile computing device,
iii. store the data in a database and transmit the data to a monitoring computer;
iv. establish a two-way communication between the monitoring computer and one or more of the mobile computing devices, and
v. assign and reassign mobile computing devices to a monitoring computer;
c. one or more databases configured to store and retrieve patient data;
d. a monitoring computer configured to:
i. receive and display patient data inputted into the mobile computing devices by one or more user members,
ii. transmit instructions through a two-way communication interface to the mobile computing devices, and one or more computing devices for use by other Care Team members configured to receive and display the electronic medical record of a patient;
wherein the system is configured to allow a non-licensed care team member on the mobile computing device to perform tasks as directed by a remote licensed care team member on the monitoring computer.

13. The system of claim 12, wherein the graphics are interactive menus, forms, and interfaces relating to the online status of care team members, alerts for incoming messages, instructions for the care team provider, status of the patient, and whether the care team member is in directing mode or observing mode.

14. The system of claim 12, wherein another care team member can observe, on the monitoring computer, the interaction between the licensed care team member and the non-licensed care team member.

15. The system of claim 12, wherein the one or more mobile computing devices is in a non-hospital location.

16. The system of claim 12, wherein the one or more mobile computing devices is in a care facility of a patient.

17. The system of claim 12, wherein the one or more mobile computing devices is in a patient's home.

18. The system of claim 12, wherein the one or more computing devices is in a non-hospital location.

19. The system of claim 12, wherein the monitoring computer is in a non-hospital location.

20. A system for enabling a clinician to monitor the health status of a patient situated in a non-hospital location and attended by a non-licensed worker, the system configured to execute the method of claim 1.
